(19) United States
(12) Patent Application Publication
(10) Pub. No.: US 2006/0184034 A1
(43) Pub. Date: Aug. 17, 2006

(54) Ultrasonic probe with an integrated display, tracking and pointing devices

(76) Inventors: Ronen Haim, Ramat Gan (IL); Esther Wischniewski, Berlin (DE); Uri Perets, Beer Sheva (IL); Valery Safran, Cfar Saba (IL)

Correspondence Address: RONEN HAIM, 21 HERUT ST, RAMAT GAN, 52541, IL

(21) Appl. No.: 11/340,552

(22) Filed: Jan. 27, 2006

Related U.S. Application Data
(60) Provisional application No. 60/647,029, filed on Jan. 27, 2005.

(51) Int. Cl. A61B 8/14

(52) U.S. Cl. 600/459

(57) ABSTRACT

An Ultrasonic probe with integrated devices and functionalities for use in a wide range of medical applications. The present invention supports vascular and arterial access procedures. The apparatus consist of the following: ultrasonic probe, several optional displays, tracking and pointing devices. All devices are integrated together with special probe housing. The probe includes numerous ultrasonic transducers arrays, disposed on the special housing. Display devices, such as LCD, TFT or other, are mounted on the probe housing on either-side, and displays an image or other information (data) from the system. All screens can be integrated together or separately on probe housing. The tracking device provides information about the probe position and the pointing device assists in guiding the needle insertion direction. Beam former hardware is interfaced to transducer elements via cable or wireless. Display, tracking and pointing devices is also interfaced to relative hardware via same cable or other method.

### CROSS-REFERENCE TO RELATED APPLICATIONS

This application is related to U.S. Provisional Patent Application 60/647,029 filed 27 Jan. 2005.

### BACKGROUND OF THE INVENTION

The present invention relates to the field of ultrasonic probes and more particularly to an ultrasonic probe with an additional integrated hardware for medical applications such as vascular and arterial access procedures. The method suggested by the present invention exempts the user from handling several procedures simultaneously such as positioning the probe, observing the image on the systems screen and locating an accurate location on the surface.

Diagnosing human organs using ultrasound scanning is a well known procedure. Ultrasonic transducers based probes direct ultrasonic waves, which travel through a selected biological medium.

Reflections are obtained each time the ultrasonic waves encounter impedance variation interfaces in the biological medium, such as fat and muscle. The returned echoes are received and processed by the imaging system that adds up all scanning lines received from the transducer and provides an image. The number of scanning lines and the depth of examination control the scanning rate. Generally speaking, standard ultrasonic probes use a one dimensional (1D) transducer, wherein the transducer elements are linearly arranged. However, in some probe configurations, multi-dimensional probes (1.5D or 2D) are provided, and the transducer elements are arranged in a matrix, so as to provide 3D steering capabilities.

Conventionally, ultrasonic probes are connected to a system, which is responsible for the processing of electrical signals produced by the probe transducer. The system performs an image capture or rendering operation, using data from the region being scanned, and the obtained images are produced by the synthesizing of information based on a number of different parameters, e.g., the transducer geometry, the number of scanning lines, the depth of examination and the transducer frequency.

The scanning process is usually performed manually. While operating a probe on a surface, the user has to use a screen, which is located far from the probe or the desired scanned area. The user must exercise few simultaneous operations such as surface scanning, monitor screening and ultrasound system control. All these operations require both hands to be used as well as multiple and simultaneous equipment operation.

The operations become rather complex for an insertion of catheters into veins or arteries. Multiple attempts at penetration may result in extreme discomfort to the patient, while multiple equipment operation is still required.

There are several patents that disclose related methods and apparatuses for ultrasonic probes. U.S. Pat. No. 6,132,379 relates to a dual mode handheld ultrasonic device, for guiding a venous access catheter into a patient's peripheral vein. It provides B-mode imaging with a predetermined aperture and operating frequency which locates and displays a gray scale cross-sectional image of the target blood vessel. A single Doppler beam in a separate mode detects the same blood vessel and creates a single scanline image superimposed to such B-mode cross-sectional image. Simultaneously, the intensity of the positive Doppler shift detected by the single Doppler beam as it hits the target blood vessel activates a plurality of light emitting diode (LED) indicator lights with varying voltage requirements mounted in the scanhead. Activated LED indicator lights forms an arrow pointing inferiorly perpendicular to the target vessel which guides a physician or a paramedical professional to the precise catheter insertion spot on a patient's extremity while simultaneously viewing the target blood vessel's cross-section on the display screen/s.

Further more, U.S. Pat. No. 6,755,789 provides an apparatus for cannulation of blood vessels, and comprises a sensor assembly including two linear transducer arrays oriented perpendicularly to each other to form a “T” shape to provide substantially simultaneous ultrasound images of at least one blood vessel in a portion of a patient's body in two perpendicular planes. The apparatus may also include one or more Doppler transducer elements to transmit and receive one or more Doppler beams at an incident angle beneath one of the transducer arrays and in alignment therewith to determine blood flow direction and velocity within the at least one blood vessel. The sensor assembly may be disposed within an elongated, flexible, protective sheath and secured to a graphically marked cover to facilitate orientation of the sensor assembly on the patient and guidance of a needle towards a desired target vessel during the cannulation procedure. The cover may also include associated structure to cooperate with a reference location element to place, align and secure the sensor assembly to the patient's skin at a desired location.

None of the patents mentioned above address the need of integrating a display, a pointing device and a tracking device in the probe housing for simpler and easier usage in medical procedures.

The present invention discloses a method and an apparatus for ultrasonic scanning, using displays, a pointing device and a tracking device mounted on the probe, for simple and easy usage, exempting the user from handling several procedures simultaneously.

### SUMMARY OF THE INVENTION

The object of the invention is to provide an Ultrasonic probe with integrated devices and functionalities for use in wide range of medical application like vascular and arterial access procedures.

The present invention exempts the user from handling several procedures simultaneously such as positioning the probe, observing the image or data on the systems screen and locating an accurate location on the surface and thus allowing the user to practice a more accurate procedure.

### BRIEF DESCRIPTION OF THE DRAWINGS

These and further features and advantages of the invention will become more clearly understood in the light of the ensuing description of a preferred embodiment thereof, given by way of example only, with reference to the accompanying drawings, wherein—

FIG. 1 is the general schematic view of the ultrasonic probe with integrated devices.

FIG. 2 is the schematic view of ultrasonic arrays architecture.

FIG. 3 is the block diagram of the ultrasound system and probe.

FIG. 4 is a schematic view of the tracking and pointing devices location on the probe housing.

FIG. 5 illustrates the pointing device configuration.

### DETAILED DESCRIPTION OF THE INVENTION

Introduction

Ultrasound equipment has developed rapidly over the past 30 years and is now used routinely for numerous medical applications, for example: assessment of arterial stenosis, venous incompetence and venous thrombosis.

Ultrasound images are obtained by holding a probe on the skin surface. An ultrasonic scanner usually has a range of probes with different characteristics, e.g., a linear array probe. This produces a rectangular image which is displayed with the skin surface at the top, the vertical axis showing depth into the body and the horizontal axis showing position along the probe. When imaging blood vessels, the probe can either be placed along the vessel to produce a longitudinal scan or across the vessel to produce a transverse scan. To produce the images, the probe emits short pulses of ultra-sound, and these travel into the body from the probe. Within the soft tissues or at boundaries between them, a small proportion of the ultrasound is scattered or reflected and arrives back at the probe as an echo. The speed of ultrasound in the body is constant (1540 m/s), so the depth of any scatterer or reflector can be found from the time delay from emitting the pulse to receiving the echo. The main pulse continues deeper into the body to be scattered or reflected from deeper structures. When the echoes from one pulse have died down, the next pulse is emitted from a slightly different position along the probe. In this way, it is possible to build up an image of a plane in the body, with depth into the body as the vertical axis and position along the probe as the horizontal axis.

The probe determines the frequency of the ultrasound within the pulses. Higher frequencies give better resolution and more detailed images, but the higher frequency sound loses energy more quickly as it travels through the body so the depth of penetration is less. The operator usually uses as high a frequency as possible. Ultrasound of these frequencies does not travel through air, so a layer of water-based coupling medium is used between probe and skin.

The present invention is comprised of an ultrasonic probe with integrated displays, tracking and pointing devices. Unlike the conventional probe, wherein the scanning probe and display interface are separate units and the user must manipulate the display-interface, the probe, the location and the image control, the present apparatus provides the user with many integrated options such as free hand scanning, imaging, data positioning and pointing.

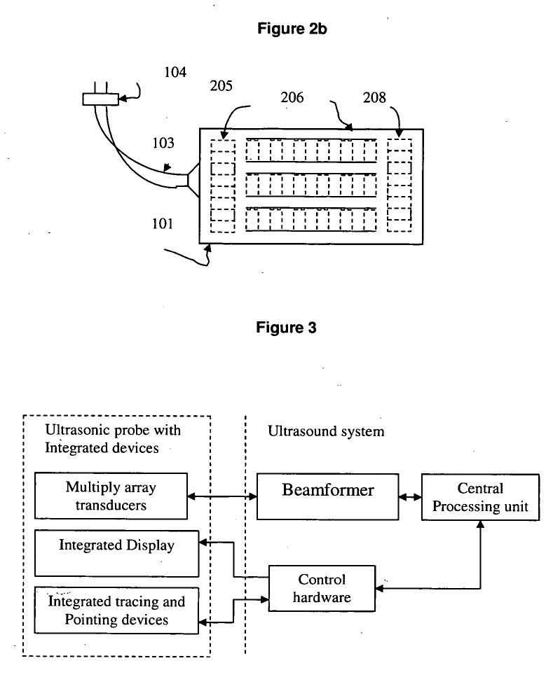

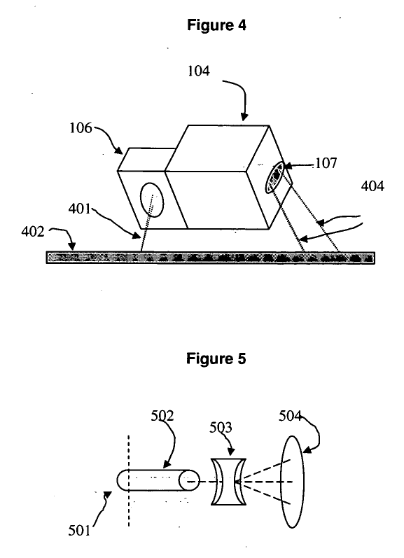

FIG. 2 illustrates the multiple transducers arrays, which are composed from standard transducers with different configuration. Multiple arrays configuration enables the receiving of 3D sliced image with minimal probe movements, reduces manual operation overhead and eases the standard probe adjustments process, together with a secured probe position to surface, allowing simultaneous operation.

Integrated displays show the data and images provided by the system, so a user can operate the apparatus in accordance to information or image viewed on display. This operational mode can be very helpful in a vascular access operation. The user can operate the probe immediately and in accordance to information obtained directly from the display. An additional accuracy and a comfortable operational environment for medical personal are achieved.

Integrated tracking device, such as the optical mouse, allows a better navigation and positioning of the probe on the surface. The pointing device enables the marking of the best possible option of entrance to the blood vessel, making an accurate and precise procedure like vascular puncture possible.

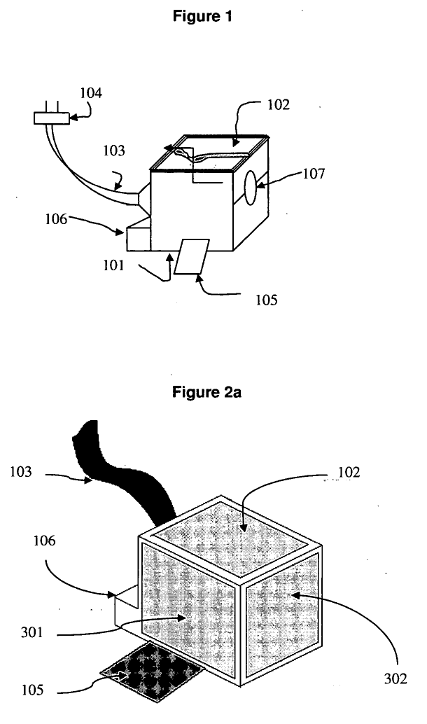

FIG. 1 illustrates the prolonged cubical shape of the probe which, helps to mount the probe on a human body. The elastic supported arms (105) which are located on both sides of the probe, secures it to the desired surface.

According to FIG. 1, the present invention is comprised of multiple transducer arrays (101) with changing configuration, which enables an advanced scanning process, resulting in 3D imaging.

The multiple transducer arrays are divided into three regions: first perpendicular array (206), parallel array (208) and second perpendicular array (206). The probe multiple transducer arrays configuration can consist of either both perpendicular arrays (208+205) or only one of them. Each array is built from two or more transducers.

The Ultrasound system beamformer—combiner (In linear diversity combining, the outputs of two coherent receiving systems are linearly combined to generate the overall system output) deals with each array independently, by switching between the arrays. When an array is switched to the beamformer, all the transducers that are on that array are operational and the system can generate a signal to every transducer within that array. When switched to another array, the Transmit/Receive (T/R) switch must be disconnected first from the previous array and all the transducers on that array are not operational. The system will generate signals to the currently connected array. The probe emits short pulses of ultra-sound, and these travel into the body from the probe. Within the soft tissues a small, proportion of the ultrasound is scattered or reflected and arrives back at the probe as an echo. The received signals are sent to the central processing unit following pre processing at the beam former. The processed image is then sent back, again via the same connector (104) to the display (102), which is integrated on the probe. The system provides processed images or other information on the display Such as an image of a scanned vascular location.

According to additional embodiment of the present invention the probe device may include more than one display. In addition to the display (102), several displays (301 and 302) can be integrated on the sidewalls of the probe housing (101). Alternatively the main display can be situated on the probe housing side. All the displays can be integrated together or separately on the probe housing, while each screen can be used forsynthetic, image representation including text, data or graphical symbols or combination. The displays may show the same image or images which provide different viewing angles or partial images when viewed together create complete picture of the scanned vascular location or other data

Integrated tracking system (106) provides the probe positioning and enables sliced indexing, as the location is depended on scanned surfaces. The tracking device, such as an optical mouse, transmits a light beam (401), which is reflected from the surface (402) and received back in the device. The positioning information data is then sent to the ultrasound system via the same connecting cable (103).

A pointing device, based on a semiconductor laser diode with low power output, is placed on the system ultrasound hardware (501) and connected to an optic fiber (502) via ultrasonic probe connecting cable. The optic fiber, integrated inside the probe housing, is connected to non-convex lens (503). The optical ray dispersed by the lens (503) is projected through the condenser to the patients' skin desired area.

In such case, the pointer device provides the blood vessel optional insertion point, which is not visually seen to the naked eye.

Each device can be integrated independently on the probe, however for a complete guided vascular access all devices need to be mounted on the probe.

Although the present invention describes in implementation for vascular and arterial access imaging device, other implementations of the present invention can be used in a variety of medical applications and procedures As well as in non vascular anatomical locations

What is claimed is:

1. An ultrasonic probe, said probe is comprised of:
a probe housing;
at least two arrays of traducers disposed on said housing, wherein the configuration of said traducers array are parallel or perpendicular;
A plurality of cables disposed within a common sheath, said cables are connected to said probe housing;
At least one display for providing processed information or image, wherein said information ore image are processed in auxiliary system.

2. The ultrasonic probe of claim 1 wherein each array is activated at a time enabling access to each transducer in the activated array;

3. The ultrasonic probe of claim 1 further including a touchpad display;

4. The ultrasonic probe of claim 1 further including a pointing display

5. The ultrasonic probe of claim 1 further including any display

6. The ultrasonic probe of claim 1 further including a tracking device which utilizes optical LED and optical mouse base sensor.

7. The ultrasonic probe of claim 1 further including a pointing device utilizing an optic fiber and a non-convex lens for light projections.

8. The ultrasonic probe of claim 1 further including a pointing device which includes an optical fiber and lens mounting;

9. The ultrasonic probe of claim 1 wherein the display is situated on the front of the probe housing.

10. The ultrasonic probe of claim 1 wherein the display is situated on the sidewall of the probe housing.

11. The ultrasonic probe of claim 1 comprising at least two displays wherein the one display is situated on the front of the probe housing and the second on the probe housing sidewall.

12. The ultrasonic probe of claim 1 comprising at least two displays wherein both display show the same image.

13. The ultrasonic probe of claim 1 comprising at least two displays wherein each display shows different image.

14. The ultrasonic probe of claim 1 comprising at least one display wherein each display shows two or more different images.

15. The ultrasonic probe of claim 1 wherein the display confirms to industry standard form and size.

16. The ultrasonic probe of claim 1 wherein the display confirms to non standard form and size.

17. The ultrasonic probe of claim 1 wherein the display confirms to any or combination of a standard or interactive (such as touchpad or pointing screen) or remotely operated.
